(19) United States
(12) Patent Application Publication
(10) Pub. No.: US 2024/0355430 A1
(43) Pub. Date: Oct. 24, 2024

(54) METHOD FOR PREDICTING PHARMACOLOGICAL EFFECTS OF NEW DRUG CANDIDATE SUBSTANCE BASED ON ARTIFICIAL INTELLIGENCE

(71) Applicant: MEDIRITA, Seoul (KR)

(72) Inventors: Young-Woo PAE, Seoul (KR); Seung-Hyun JIN, Seoul (KR)

(73) Assignee: MEDIRITA, Seoul (KR)

(21) Appl. No.: 18/685,729

(22) PCT Filed: Apr. 11, 2022
(86) PCT No.: PCT/KR2022/005220

§ 371 Date: Feb. 22, 2024

(30) Foreign Application Priority Data
Mar. 30, 2022 (KR) 10-2022-0039557

(51) Int. Cl. G16C 20/30; G16C 20/70

(52) U.S. Cl. CPC G16C 20/30; G16C 20/70

(57) ABSTRACT

Provided is a method for predicting pharmacological effects of a new drug candidate substance performed by a computing device, wherein the method may include receiving information on a new drug candidate substance, selecting a structural similarity type, which is a reference for determining the similarity between substances, preparing pharmacological effect prediction models corresponding to the selected structural similarity type from among a plurality of pharmacological effect prediction models created by structural similarity type and pharmacological class, and predicting whether the new drug candidate substance will have a pharmacological class corresponding to each of the pharmacological effect prediction models based on an output value obtained by inputting information on the new drug candidate substance into each of the prepared pharmacological effect prediction models.

### TECHNICAL FIELD

The present invention relates to a method for developing a new drug, and more specifically, to a method for predicting pharmacological effects of a newly discovered or designed new drug candidate substance.

### BACKGROUND ART

It is known that it takes a total of 15 years on average to develop a new drug, and costs 2 to 3 trillion Korean won. It takes about 6 years to discover a new drug candidate substance, and once the new drug candidate substance is discovered, it takes a very long period of time to find out through preclinical trials and clinical trials whether there are any problems when the new drug candidate substance is administered to the human body and what pharmacological effects the new drug candidate substance has. Whether the new drug candidate will have pharmacological effects that meet a researcher's goals can only be determined through clinical trials. Therefore, if it is determined that desired pharmacological effects will not be obtained, a new drug candidate substance needs to be acquired and to undergo clinical trials again, which is very costly and time-consuming.

According to the Life Intelligence Consortium (2017) recently launched in Japan, it is predicted that if artificial intelligence technology is used in the development of new drugs, the time required to develop a new drug may be shortened by about 40%, and the cost to do so may be reduced by about 50%.

### DISCLOSURE OF THE INVENTION

### Technical Problem

Provided is a method for predicting pharmacological effects of a new drug candidate substance by calculating the similarity between a newly discovered or designed new drug candidate substance with a substance for which the pharmacological effects are already known, and applying calculated results to an artificial intelligence prediction model.

The technical task to be achieved by the present embodiment is not limited to the technical task as described above, and other technical tasks may be inferred from the following embodiments.

### Technical Solution

Provided is a method for predicting pharmacological effects of a new drug candidate substance by calculating the similarity between a newly discovered or designed new drug candidate substance with a substance for which the pharmacological effects are already known, and applying calculated results to an artificial intelligence prediction model.

### Advantageous Effects

It is possible to predict whether a new drug candidate substance will have pharmacological effects that meet a researcher's goals before clinical trials by using an artificial intelligence prediction model. There is an effect of drastically reducing the cost and time of unnecessary clinical trials by conducting actual clinical trials based on predicted results.

### BRIEF DESCRIPTION OF THE DRAWINGS

FIG. 1 shows a flowchart of a method for predicting pharmacological effects of a new drug candidate substance in a computing device, according to one embodiment.

FIG. 2 shows a concept of creating a pharmacological effect prediction model by type of structural similarity and, further, by type of pharmacological class (PC), according to one embodiment.

FIG. 3 shows a flowchart of a method for creating a pharmacological effect prediction model, according to one embodiment.

FIG. 4 is a flowchart of a method for predicting pharmacological effects of a new drug candidate substance by using a pharmacological effect prediction model, according to an embodiment.

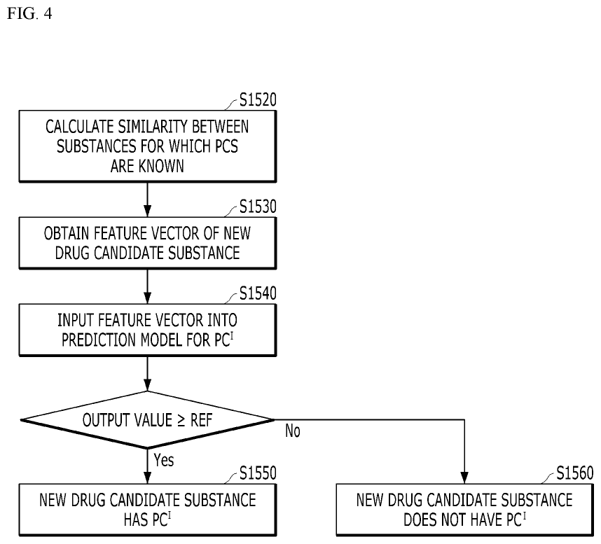

FIG. 5 illustrates a concept of obtaining an output value of each of pharmacological effect prediction models created by type of PC by inputting information on a new drug candidate substance, according to an embodiment.

FIG. 6 shows an input and an output of an artificial neural network, according to one embodiment.

FIG. 7 shows a screen on which a value for the probability that a new drug candidate has a specific PC is output, according to one embodiment.

### BEST MODE FOR CARRYING OUT THE INVENTION

A method for predicting pharmacological effects of a new drug candidate substance performed by a computing device includes receiving information on a new drug candidate substance, selecting a structural similarity type, which is a reference for determining the similarity between substances, preparing pharmacological effect prediction models corresponding to the selected structural similarity type from among a plurality of pharmacological effect prediction models created by structural similarity type and pharmacological class, and predicting whether the new drug candidate substance will have a pharmacological classification corresponding to each of the pharmacological effect prediction models based on an output value obtained by inputting information on the new drug candidate substance into each of the prepared pharmacological effect prediction models, wherein the structural similarity type is further classified according to which calculation method between a Dice similarity calculation method and a Tanimoto similarity calculation method is to be applied, whether a Bemis-Murcko scaffold is applied, and whether a hydrogen atom bond is applied, each of the plurality of pharmacological effect prediction models created by structural similarity type and pharmacological class is created based on machine learning using substances already known whether to have a specific pharmacological class, in each of the pharmacologic effect prediction models, a binary vector obtained by using a threshold value for binarization is input as a feature vector of the new drug candidate substance to a similarity calculated according to the selected structural similarity type between the new drug candidate substance and the already known substances, the predicting step predicts, if a first output value obtained by inputting the feature vector into a first pharmacological effect prediction model corresponding to a first pharmacological class is greater than or equal to a reference value, that the new drug candidate substance has the first pharmacological class, if the first output value is less than the reference value, that the new drug candidate substance does not have the first pharmacological class, if a second output value obtained by inputting the feature vector into a second pharmacological effect prediction model corresponding to a second pharmacological class is greater than or equal to the reference value, that the new drug candidate substance has the second pharmacological class, and if the first output value is less than the reference value, that the new drug candidate substance does not have the first pharmacological class.

The method for predicting pharmacological effects of a new drug candidate substance performed by a computing device may further include creating the plurality of pharmacological effect prediction models by structural similarity type and pharmacological class, wherein the creating of the plurality of pharmacological effect prediction models includes creating a pharmacological effect prediction model corresponding to a specific pharmacological class, wherein the creating of the pharmacological effect prediction model corresponding to the specific pharmacological class may further include creating a two-dimensional adjacency matrix in which substances known whether to have the specific pharmacological class are disposed on each of a horizontal axis and a vertical axis, and similarity between two substances calculated on the basis of one of the structural similarity types is displayed at a point at which the horizontal axis and the vertical axis intersect, obtaining a binary adjacency matrix by converting the similarity to 1 if the similarity is greater than or equal to the threshold value and at least one of the two substances is known to have the specific pharmacological class, and by converting the similarity to 0 if the similarity is less than the threshold value or if both the two substances are known not to have the specific pharmacological class, obtaining values listed in each of rows constituting the binary adjacency matrix as a feature vector of the known substances corresponding to each of the rows, and creating a pharmacological effect prediction model corresponding to one of the structural similarity types and the specific pharmacological class by applying the feature vector of the known substances and whether each of the known substances has the specific pharmacological class as an input and an output, respectively to perform training.

The creating of the pharmacological effect prediction model corresponding to the specific pharmacological class may further include reducing the dimension of the binary adjacency matrix by applying a principle component analysis (PCA) technique, wherein the feature vector of the known substances may be obtained from the dimensionally reduced binary adjacency matrix.

The method for predicting pharmacological effects of a new drug candidate substance performed by a computing device may further include obtaining the feature vector of the new drug candidate substance that is input into the pharmacological effect prediction model, wherein the obtaining of the feature vector of the new drug candidate substance may include calculating similarity between the new drug candidate substance and the known substances on the basis of the selected structural similarity type, and obtaining, as the feature vector of the new drug candidate substance, a binary vector obtained by converting the similarity to 1 if the similarity is greater than or equal to the threshold value, and by converting the similarity to 0 if the similarity is less than the threshold value.

The method for predicting pharmacological effects of a new drug candidate substance performed by a computing device may further include obtaining the feature vector of the new drug candidate substance that is input into the pharmacological effect prediction model, wherein the obtaining of the feature vector of the new drug candidate substance includes calculating similarity between the new drug candidate substance and the known substances on the basis of the selected structural similarity type, obtaining a binary vector by converting the similarity to 1 if the similarity is greater than or equal to the threshold value, and by converting the similarity to 0 if the similarity is less than the threshold value, and obtaining, as the feature vector of the new drug candidate substance, a result obtained by performing vector product on an eigen vector derived by applying the principle component analysis (PCA) technique by the binary vector.

### MODE FOR CARRYING OUT THE INVENTION

Below, some embodiments will be described clearly and in detail with reference to the accompanying drawings so that those of ordinary skill in the art (hereinafter, referred to as those skilled in the art) to which the present invention belongs may easily practice the present invention.

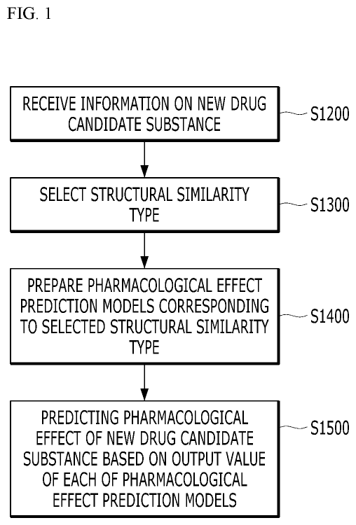

FIG. 1 shows a flowchart of a method for predicting pharmacological effects of a new drug candidate substance in a computing device, according to one embodiment.

The computing device may include at least one processor and at least one memory. The processor may include a central processing unit (CPU), a microprocessor, a graphic processing unit (GPU), a digital signal processor (DSP), or a micro controller unit (MCU). The memory may include a volatile memory, such as a dynamic random access memory (DRAM) and a static random access memory (SRAM), and a non-volatile memory, such as a flash memory, a read only memory (ROM), a phase-change random access memory (PRAM), a magnetic random access memory (MRAM), a resistive random access memory (ReRAM) and a ferroelectrics random access memory (FRAM). According to one embodiment, the computing device is composed of physically separated separate devices and the separate devices may transmit and receive data through a wired or wireless communication interface. The computing device may be in the form of a PC or a laptop and may include a server device, but is not limited thereto.

In Step S1200, the computing device may receive information on a new drug candidate substance.

The new drug candidate material is an unknown novel material and is a material whose pharmacological effects are intended to be predicted. The information on the new drug candidate substance may be received in the form of a drug structure picture file such as a structure-data file (SDF), a string expressed according to a simplified molecular-input line-entry system (SMILES) of a substance, or a chemical name (e.g., International Union of Pure and Applied Chemistry (IUPAC)).

As an example, the received information on the new drug candidate substance may include N-(4-hydroxyphenyl) acetamide, CCOC1=C2C3=C(CCCN3)C(═O)[NH]C2=CC(CN2CCOCC2)=C1, a picture file such as a picture below, or the like.

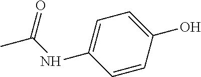

In Step S1300, a structural similarity type may be selected.

The structural similarity type is a reference for determining similarity between substances, and is for determining on what basis the similarity between substances should be determined. A user may select the structural similarity type.

According to one embodiment, types of structural similarity may be broadly classified into a Dice similarity based on a dice coefficient and a Tanimoto similarity based on a Tanimoto coefficient according to a similarity calculation method (reference link: https://www.ccdc.cam.ac.uk/support-and-resources/support/case/?caseid=899a6a77-e3 79-4981-84f4-07de67f39016).

The Tanimoto similarity is a method of calculating similarity based on a ratio of the number of common features among the total number of features of both molecules, and the Dice similarity is a method of calculating similarity based on a ratio of the number of common features among values obtained by averaging the total number of features of each molecule.

According to one embodiment, methods for calculating similarity (Dice similarity and Tanimoto similarity) may be further classified again depending on whether a ‘Bemis-Murcko scaffold’ is applied and whether a ‘Add hydrogen atoms’ is applied. The ‘Bemis-Murcko scaffold’ is a method for evaluating similarity on the basis of a chemical structure expressed around a main ring structure constituting a molecule through a method for removing a side chain atom in a molecular structure. The ‘Add hydrogen atoms’ is a method for evaluating similarity by combining a hydrogen atom to a compound. Since the stability and form of a chemical structure differ when a hydrogen atom is combined through hydrogen bonding or not, a hydrogen donor count and a hydrogen acceptor count are known to be one of main factors in identifying properties of a compound.

Since the ‘Bemis-Murcko scaffold’ and the ‘Add hydrogen atoms’ are not alternative relationships, both the ‘Bemis-Murcko scaffold’ and the ‘Add hydrogen atoms’ may be applied for a similarity calculation, either the ‘Bemis-Murcko scaffold’ or the ‘Add hydrogen atoms’ may be applied for a similarity calculation, or similarity may be calculated without applying both the ‘Bemis-Murcko scaffold’ and the ‘Add hydrogen atoms.’ Table 1 below shows eight structural similarity types according to one embodiment.

TABLE 1

| | | Bemis- | Add | |
|---|---|---|---|---|
| | Similarity | Murcko | hydrogen | |
| | calculation | scaffold | atoms | Structural |
| # | method | application | application | similarity type |
| 1 | Dice similarity | X | ◯ | Dice_AddHs |
| 2 | Dice similarity | X | X | Dice |
| 3 | Dice similarity | ◯ | ◯ | Dice_Murcko_AddH s |
| 4 | Dice similarity | ◯ | X | Dice_Murcko |
| 5 | Tanimoto similarity | X | ◯ | Tanimoto_AddHs |
| 6 | Tanimoto similarity | X | X | Tanimoto |
| 7 | Tanimoto similarity | ◯ | ◯ | Tanimoto_Murcko_AddHs |
| 8 | Tanimoto similarity | ◯ | X | Tanimoto_Murcko |

Referring to Table 1, the structural similarity types may be further classified into 8 types (Dice similarity w H+, Dice similarity w/o H+, Dice similarity Murcko scaffold w H+, Dice similarity Murcko scaffold w/o H+, Tanimoto similarity w H+, Tanimoto similarity w/o H+, Tanimoto similarity Murcko scaffold w H+, and Tanimoto similarity Murcko scaffold w/o H+) according to which of the two similarity calculation methods (Dice vs Tanimoto) is to be applied, whether the ‘Bemis-Murcko scaffold’ is applied, and whether the ‘Add hydrogen atoms’ is applied. The similarity between substances may be calculated differently depending on the selection of a structural similarity type. The method for predicting pharmacological effects of the present invention is based on a premise that if structural similarity between two substances is high, the two substances have similar pharmacological effects, SO that a selected structural similarity type is an important reference in a process of predicting pharmacological effects of a new drug candidate substance and in a process of creating a pharmacological effect prediction model through machine learning.

In Step S1400, the computing device may prepare pharmacological effect prediction models corresponding to the structural similarity type selected in Step S1300 among a plurality of pharmacological effect prediction models. According to one embodiment, pharmacological effect prediction models may be loaded into a memory of the computing device.

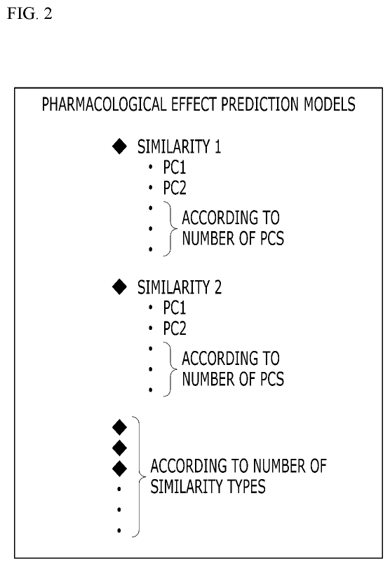

A pharmacological effect prediction model may be created by structural similarity, and may be created for each pharmacological class (PC) in one structural similarity category (see FIG. 2). According to the definition of the United States Food and Drug Administration (FDA), the “Pharmacological Class” refers to a class of an active moiety group defined on the basis of three attributes of a mechanism of Action (MOA), a physiologic effect (PE), and a chemical infrastructure (CS), and the United States Food and Drug Administration requires that a clinically meaningful class among MOA, PE, and CS reported for a specific drug be included in prescription information of the drug. That is, a pharmacological class is pharmacological effects of a specific material, and a specific pharmacological class may mean a specific pharmacological effect.

As an example, types of pharmacological classes (hereinafter, PC) may include enzyme inhibitors, anti-infective agents, central nervous system agents, neurotransmitter agents, etc., and may be more than 1,000.

TABLE 2
Type of PC

Enzyme Inhibitors
Anti-Infective Agents
Central Nervous System Agents
Neurotransmitter Agents
Antineoplastic Agents
Peripheral Nervous System Agents
Cardiovascular Agents
Cytochrome P-450 Enzyme Inhibitors
Central Nervous System Depressants
Anti-Bacterial Agents
Sensory System Agents
Analgesics
Anti-Inflammatory Agents
Cytochrome P-450 CYP3A Inhibitors
P-Glycoprotein Inhibitors
Psychotropic Drugs
Antirheumatic Agent
Amides
Antihypertensive Agents
Adrenergic Agents
. . .

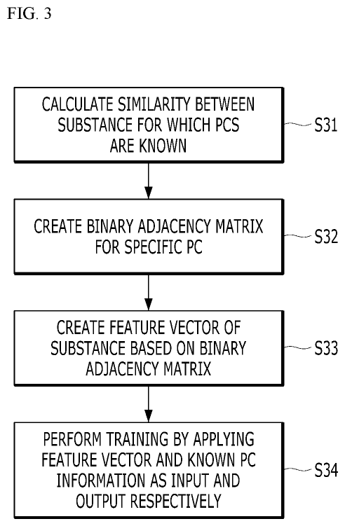

Each of the plurality of pharmacological effect prediction models corresponds 1:1 to a specific structural similarity PC, and a specific type, a specific pharmacological class. That is, the plurality of pharmacological effect prediction models are created as many as the number of M×N (M: number of structural similarity types, N: number of PC types), and one of the pharmacological effect prediction models is a model corresponding to a specific structural similarity and a specific PC (e.g., a model having the similarity type of Dice_AddHs and the PC of an enzyme inhibitor). A pharmacological effect prediction model is created on the basis of machine learning using substances for which the PC information is already known, and when the model receives data on a substance, the model may output probability that the substance will have a specific PC. A method for creating a pharmacological effect prediction model will be described in detail later with reference to FIG. 3. In Step S1500, the computing device may predict pharmacological effects of a new drug candidate substance by inputting data on a new drug candidate substance into at least one pharmacological effect prediction models loaded in Step S1400 to obtain a probability value for each of PCs. Since the pharmacological effect prediction model is created by PC, it is possible to predict whether a new drug candidate substance will have a PC corresponding to a current pharmacological effect prediction model based on an output value of the current pharmacological effect prediction model.

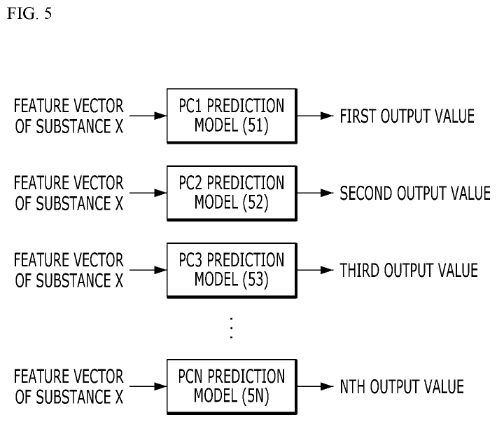

Referring to FIG. 5, the computing device inputs a new drug candidate substance X into a prediction model 51 for a PC1 to obtain a first output value. If the first output value is greater than or equal to a reference value, it is possible to predict that the new drug candidate substance X has the PC1. If otherwise, it is possible to predict that the new drug candidate substance X does not have the PC1.

The computing device inputs the new drug candidate substance X into a prediction model 52 for a PC2 to obtain a second output value. The computing device may predict that if the second output value is greater than or equal to the reference value, the new drug candidate substance X has the PC2. If otherwise, the computing device may predict that the new drug candidate substance X does not have the PC2.

The computing device inputs the new drug candidate substance X into a prediction model 53 for a PC3 to obtain a third output value. The computing device may predict that if the third output value is greater than or equal to the reference value, the new drug candidate substance X has the PC3. If otherwise, the computing device may predict that the new drug candidate substance X does not have the PC3.

The computing device inputs the new drug candidate substance X into a prediction model 5N for a PCN to obtain an N-th output value. The computing device may predict that if the N-th output value is greater than or equal to the reference value, the new drug candidate substance X has the PCN. If otherwise, the computing device may predict that the new drug candidate substance X does not have the PCN. According to one embodiment, each output value may be a value between 0 and 1, and the reference value may be 0, but the embodiment is not limited thereto.

FIG. 3 shows a flowchart of a method for creating a pharmacological effect prediction model, according to one embodiment.

The flowchart of FIG. 3 may include a method for creating a single pharmacological effect prediction model corresponding to a specific structural similarity and a specific PC.

In Step S31, the computing device may calculate structural similarity between substances for which the PC information is already known (hereinafter, substances for which the PC is known). A reference for calculating structural similarity, i.e., a structural similarity type, may be one of the eight types in Table 1, but is not limited thereto. A method for calculating similarity between substances according to a specific structural similarity type is widely known to those skilled in the art, and thus, a detailed description thereof will be omitted (reference link: https://www.ccdc.cam.ac.uk/support-and-resources/support/case/?caseid=899a6a77-e3 79-4981-84f4-07de67f39016).

The relationship between a substance and a PC may be obtained from FDA/BioPortal (https://bioportal.bioontology.org), MeSH (https://www.ncbi.nlm.nih.gov/mesh), ChEBI (https://www.ebi.ac.uk/chebi/init.do), etc. The number of substances for which the PCs are already known is approximately 2,000 to 3,000, and the number thereof may continue to increase. Table 3 below shows some substances of the substances for which the PCs are already known. Referring to Table 3 below, the substance CID77999 is known to have PCs of Thiazolidinediones, PPAR gamma, Peroxisome Proliferator-activated Receptor Activity, Peroxisome Proliferator Receptor gamma Agonist, and Hypoglycemic Agents. The substance CID4829 is known to have Pcs of Thiazolidinediones, PPAR gamma, PPAR alpha, Peroxisome Proliferator-activated Receptor Activity, Hypoglycemic Agents, Peroxisome Proliferator Receptor alpha Agonist, and Peroxisome Proliferator Receptor gamma Agonist.

TABLE 3

| | ID of | | |
|---|---|---|---|
| | substance | SMILE of substance | PC Name |
| 1 | CID77999 | CN(CCOC1═CC═C(C═C1)CC2C(═O)NC(═O)S2)C3═CC═CC═N3 | Thiazolidinediones |
| 2 | CID77999 | CN(CCOC1═CC═C(C═C1)CC2C(═O)NC(═O)S2)C3═CC═CC═N3 | PPAR gamma |
| 3 | CID77999 | CN(CCOC1═CC═C(C═C1)CC2C(═O)NC(═O)S2)C3═CC═CC═N3 | Peroxisome |
| | | | Proliferator- |
| | | | activated |
| | | | Receptor Activity |
| 4 | CID77999 | CN(CCOC1═CC═C(C═C1)CC2C(═O)NC(═O)S2)C3═CC═CC═N3 | Peroxisome |
| | | | Proliferator |
| | | | Receptor gamma |
| | | | Agonist |
| 5 | CID77999 | CN(CCOC1═CC═C(C═C1)CC2C(═O)NC(═O)S2)C3═CC═CC═N3 | Hypoglycemic |
| | | | Agents |
| 6 | CID4829 | CCC1═CN═C(C═C1)CCOC2═CC═C(C═C2)CC3C(═O)NC(═O)S3 | Thiazolidinediones |
| 7 | CID4829 | CCC1═CN═C(C═C1)CCOC2═CC═C(C═C2)CC3C(═O)NC(═O)S3 | PPAR gamma |
| 8 | CID4829 | CCC1═CN═C(C═C1)CCOC2═CC═C(C═C2)CC3C(═O)NC(═O)S3 | PPAR alpha |
| 9 | CID4829 | CCC1═CN═C(C═C1)CCOC2═CC═C(C═C2)CC3C(═O)NC(═O)S3 | Peroxisome |
| | | | Proliferator- |
| | | | activated |
| | | | Receptor Activity |
| 10 | CID4829 | CCC1═CN═C(C═C1)CCOC2═CC═C(C═C2)CC3C(═O)NC(═O)S3 | Hypoglycemic |
| | | | Agents |
| 11 | CID4829 | CCC1═CN═C(C═C1)CCOC2═CC═C(C═C2)CC3C(═O)NC(═O)S3 | Peroxisome |
| | | | Proliferator |
| | | | Receptor alpha |
| | | | Agonist |
| 12 | CID4829 | CCC1═CN═C(C═C1)CCOC2═CC═C(C═C2)CC3C(═O)NC(═O)S3 | Peroxisome |
| | | | Proliferator |
| | | | Receptor gamma |
| | | | Agonist |

The computing device may create an adjacency matrix including similarity information between substances for which the PCs are known. The adjacency matrix may be a two-dimensional matrix in which substances for which the PCs are known are disposed on each of a horizontal axis and a vertical axis, and a similarity value calculated based on one of the structural similarity types is displayed at a point at which the horizontal axis and the vertical axis intersect.

According to one embodiment, the adjacency matrix may have a size of K×K (where K may be the number of substance for which the PCs are known). For example, if there are 2886 types of substances for which the PCs are known, an adjacency matrix corresponding to a specific structural similarity type has a size of 2886×2886. An adjacency matrix is created separately for each structural similarity reference, and according to the structural similarity types in Table 1, eight adjacency matrices may be created.

Table 4 below shows a portion of an adjacent matrix in which similarity between predetermined substances for which the PCs are known is calculated based on a structural similarity type ‘Dice,’ according to one embodiment.

TABLE 4

| | Substance | Substance | Substance | Substance | Substance | Substance | Substance | Substance | Substance | Substance |
|---|---|---|---|---|---|---|---|---|---|---|
| | 0 | 1 | 2 | 3 | 4 | 5 | 6 | 7 | 8 | 9 |
| 0 | 1 | 0.75 | 0.299 | 0.272 | 0.373 | 0.075 | 0.039 | 0.325 | 0.3 | 0.34 |
| 1 | 0.75 | 1 | 0.272 | 0.659 | 0.363 | 0.075 | 0.039 | 0.287 | 0.282 | 0.283 |
| 2 | 0.299 | 0.272 | 1 | 0.347 | 0.306 | 0.044 | 0.038 | 0.356 | 0.306 | 0.298 |
| 3 | 0.272 | 0.659 | 0.347 | 1 | 0.296 | 0.088 | 0.095 | 0.368 | 0.352 | 0.335 |
| 4 | 0.373 | 0.363 | 0.306 | 0.296 | 1 | 0.066 | 0.026 | 0.335 | 0.321 | 0.352 |
| 5 | 0.075 | 0.075 | 0.044 | 0.088 | 0.066 | 1 | 0.22 | 0.067 | 0.05 | 0.05 |
| 6 | 0.039 | 0.039 | 0.038 | 0.095 | 0.026 | 0.22 | 1 | 0.085 | 0.105 | 0.106 |
| 7 | 0.325 | 0.287 | 0.356 | 0.368 | 0.335 | 0.067 | 0.085 | 1 | 0.306 | 0.316 |
| 8 | 0.3 | 0.282 | 0.306 | 0.352 | 0.321 | 0.05 | 0.105 | 0.306 | 1 | 0.534 |
| 9 | 0.34 | 0.283 | 0.298 | 0.335 | 0.352 | 0.05 | 0.106 | 0.316 | 0.534 | 1 |

In Step S32, the computing device may create a binary adjacency matrix for a specific PC based on the similarity matrix. When determining based on a specific structural similarity type, the binary adjacency matrix displays ‘1’ if there is a degree of association (similarity) between two substances, and displays ‘0’ if otherwise, but displays ‘1’ only if at least one of the two substances has a specific PC. A threshold (TH) is used for binarization of a value of the adjacency matrix created in Step S31. If a similarity value is greater than or equal to the threshold value, ‘1’ is displayed, and if otherwise, ‘0’ is displayed, but if none of the two substances has a specific PC, the value of a binary adjacency matrix for the specific PC may be ‘0.’

For example, if only an adjacency matrix between Substance 0 and Substance 3 in Table 4 above is extracted, it is as shown in Table 5 below, and if the threshold value is set to 0.5, a binarized similarity matrix (S) as shown in Table 6 may be obtained.

TABLE 5

| | Substance 0 | Substance 1 | Substance 2 | Substance 3 |
|---|---|---|---|---|
| Substance 0 | 1 | 0.75 | 0.299 | 0.272 |
| Substance 1 | 0.75 | 1 | 0.272 | 0.659 |
| Substance 2 | 0.299 | 0.272 | 1 | 0.347 |
| Substance 3 | 0.272 | 0.659 | 0.347 | 1 |

TABLE 6

| | Substance 0 | Substance 1 | Substance 2 | Substance 3 |
|---|---|---|---|---|
| Substance 0 | 1 | 1 | 0 | 0 |
| Substance 1 | 1 | 1 | 0 | 1 |
| Substance 2 | 0 | 0 | 1 | 0 |
| Substance 3 | 0 | 1 | 0 | 1 |

A PC^{N}ᵢⱼ of Equation 1 below is a value positioned in an i row and a j column of a binary adjacency matrix for a specific PC^{N}, and Sᵢⱼ is a value positioned in an i row and a j column of a binarized similarity adjacency matrix.

$${{PC}^{N}}_{ij} = 1 ( when {S}_{ij} = 1 and ( Substance i or Substance j has a current {PC}^{N} ) \qquad [ Equation 1 ]$$

$${{PC}^{N}}_{ij} = 0 , otherwise$$

$$( {S}_{ij} = 1 ( when similarity of Substance i and Substance j >= threshold value ) , {S}_{ij} = 0 ( otherwise )$$

$$( N is an integer greater than 0 and less than or equal to the total number of PC types )$$

For convenience of description, assuming that the known PC for each of Substance 0 to Substance 3 is as shown in Table 7 below, a method for creating a binary adjacency matrix by PC when similarity between substances is as shown in Table 5 above will be described. Each of ‘Ga,’ ‘Na,’ ‘Da,’ and ‘Ra’ is one among about 2,000 PCs and may be one of the PCs in Table 1.

TABLE 7

| Type of substance | Known PC |
|---|---|
| Substance 0 | Ga, Na, Da |
| Substance 1 | Da, Ra |
| Substance 2 | Na, Ra |
| Substance 3 | Ga, Na |

In this embodiment, a binary adjacency matrix created for each of the PCs ‘Ga,’ ‘Na,’ ‘Da,’ and ‘Ra’ according to Equation 1 is as shown in Tables 71, 72, 73, and 74 below. Table 71 shows the binary adjacency matrix for the PC ‘Ga,’ Table 72 shows the binary adjacency matrix for the PC ‘Na,’ Table 73 shows the binary adjacency matrix for the PC ‘Da,’ and Table 74 shows the binary adjacency matrix for the PC ‘Ra.’

TABLE 71

| Type of | | | | |
|---|---|---|---|---|
| substance | Substance 0 | Substance 1 | Substance 2 | Substance 3 |
| Substance 0 | 1 | 1 | 0 | 0 |
| Substance 1 | 1 | 0 | 0 | 1 |
| Substance 2 | 0 | 0 | 0 | 0 |
| Substance 3 | 0 | 1 | 0 | 1 |

TABLE 72

| Type of | | | | |
|---|---|---|---|---|
| substance | Substance 0 | Substance 1 | Substance 2 | Substance 3 |
| Substance 0 | 1 | 1 | 0 | 0 |
| Substance 1 | 1 | 0 | 0 | 1 |
| Substance 2 | 0 | 0 | 1 | 0 |
| Substance 3 | 0 | 1 | 0 | 1 |

TABLE 73

| Type of | | | | |
|---|---|---|---|---|
| substance | Substance 0 | Substance 1 | Substance 2 | Substance 3 |
| Substance 0 | 1 | 1 | 0 | 0 |
| Substance 1 | 1 | 1 | 0 | 1 |
| Substance 2 | 0 | 0 | 0 | 0 |
| Substance 3 | 0 | 1 | 0 | 0 |

TABLE 74

| Type of | | | | |
|---|---|---|---|---|
| substance | Substance 0 | Substance 1 | Substance 2 | Substance 3 |
| Substance 0 | 0 | 1 | 0 | 0 |
| Substance 1 | 1 | 1 | 0 | 1 |
| Substance 2 | 0 | 0 | 1 | 0 |
| Substance 3 | 0 | 1 | 0 | 0 |

In Step S33, the computing device may create a feature vector of a substance required for training an AI model based on the binary adjacency matrix. According to one embodiment, a feature vector of Substance i is a value of a row corresponding to Substance i on the binary adjacent matrix, i.e., an i-th row. According to one embodiment, in the examples of Tables 71 to 74 above, feature vectors of Substance 0, Substance 1, Substance 2, and Substance 3 input into a pharmacological effect prediction model for the PC ‘Ga’ are (1, 1, 0, 0), (1, 0, 0, 1), (0, 0, 0, 0), and (0, 1, 0, 1), respectively. According to one embodiment, feature vectors of Substance 0, Substance 1, Substance 2, and Substance 3 input into a pharmacological effect prediction model for the PC ‘Na’ are (1, 1, 0, 0), (1, 0, 0, 1), (0, 0, 1, 0), and (0, 1, 0, 1), respectively. According to one embodiment, feature vectors of Substance 0, Substance 1, Substance 2, and Substance 3 input into a pharmacological effect prediction model for the PC ‘Da’ are (1, 1, 0, 0), (1, 1, 0, 1), (0, 0, 0, 0), and (0, 1, 0, 0), respectively. According to one embodiment, feature vectors of Substance 0, Substance 1, Substance 2, and Substance 3 input into a pharmacological effect model for the PC ‘Ra’ are (0, 1, 0, 0), (1, 1, 0, 1), (0, 0, 1, 0), and (0, 1, 0, 0), respectively.

However, as the number of substances increases, the adjacency matrix created in Step S32 becomes very high-dimensional and the size of a feature vector also becomes very large. For example, if there are 2886 types of substances for which the PCs are known, an adjacency matrix corresponding to a selected structural similarity type has a size of 2886×2886, and a feature vector also has a size of 1×2886. As the size of a feature vector increases, the distance between artificial intelligence data points increases exponentially, and due to a sparse structure, prediction reliability may be lower than that of a model trained in relatively few dimensions. In addition, as the number of feature vectors for training increases, the likelihood of high correlation between individual feature vectors increases, prediction performance of a model may be degraded. In addition, if the size of a feature vector becomes too large, resources required for training also increase, so that there are disadvantage in that a high-specification computing device is required and a very long period of time of training is required.

According to one embodiment, the computing device may reduce a dimension of an adjacency matrix for a specific PC and create a feature vector based on the reduced matrix. For example, the computing device may reduce a dimension by applying a principal component analysis (PCA) to an adjacent matrix for a specific PC obtained in Step S32. In this embodiment, a specific substance may have a feature vector of size 1×2, which is composed of Principal Component 1 (PC1) and Principal Component 2 (PC2). In the case of a binary adjacency matrix for the PC ‘Ra’ of Table 74, a dimension may be reduced through PCA transformation and feature vectors of Substance 0, Substance 1, Substance 2, and Substance 3 become (0.8, 0), (0.5, 0.5), (0, 0), and (0, 0.4), respectively. Table 8 below shows a matrix obtained by subjecting the adjacency matrix of Table 74 to PCA transformation.

TABLE 8

| Type of substance | PC1 | PC2 |
|---|---|---|
| Substance 0 | 0.8 | 0 |
| Substance 1 | 0.5 | 0.5 |
| Substance 2 | 0 | 0 |
| Substance 3 | 0 | 0.4 |

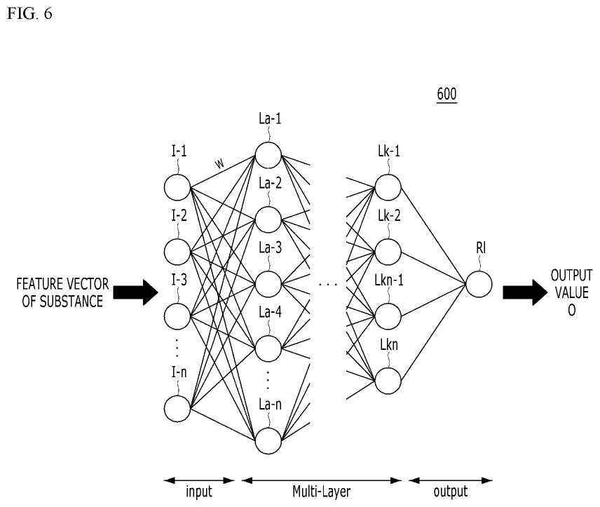

In Step S34, the computing device may obtain a pharmacological effect prediction model by PC by performing machine learning using the feature vectors by PC obtained in Step S33. The pharmacological effect prediction model by PC refers to an artificial neural network trained by a machine learning or deep learning technique to determine pharmacological effects of a specific substance. For example, the computing device may deep-train a pharmacological effect prediction model by using feature vectors obtained for a specific PC as input data. According to a representative embodiment, referring to FIG. 6, an artificial neural network 600 may include input layers I-1 to I-n, hidden layers La-1 to La-n, and Lk-1 to Lk-n, and an output layer RI. The hidden layers La-1 to La-n, and Lk-1 to Lk-n may be formed of k layers each including n nodes. Nodes of the artificial neural network 600 may be connected through synapses having weights. At least one of the nodes of the artificial neural network 600 may have a nonlinear activation function defined. The computing device may receive a feature vector of a substance as input data to perform an operation on the input data using weight values of the artificial neural network 600 and an operator/operand of the node and obtain output data O.

The computing device may perform training on the artificial neural network 600. As learning data used for training, data of substances for which the PCs are already known may be used. For example, feature vectors (Step S33 in FIG. 3) of substances for which the PCs are already known may be applied to the input data of the artificial neural network 600, and whether there is a specific PC already known may be applied to the output data. Training of the artificial neural network 600 may be performed such that a difference between an output value of the artificial neural network 600 and a value according to whether there is an actual PC is reduced. Since a process in which the artificial neural network 600 performs an operation on input data I and obtains output data O is obvious to those skilled in the art, a detailed description thereof will be omitted.

In the embodiments of Tables 6 to 7, Table 9 below may be used as an input and an output for training a prediction model for the PC ‘Ga.’ An output value through an operation of the prediction model for the PC ‘Ga’ is a value between 0 and 1, and training may be performed to reduce a difference between the output value and an output value in the right column of Table 9. According to one embodiment, when dimension reduction using PCA is performed in Step S33, a dimensionally reduced feature vector may be used as input data.

TABLE 9

| Type of | Training input | Training output |
|---|---|---|
| substance | data (value) | data (value) |
| Substance 0 | (1 1 0 0) | 1 |
| Substance 1 | (1 0 0 1) | 0 |
| Substance 2 | (0 0 0 0) | 0 |
| Substance 3 | (0 1 0 1) | 1 |

In the embodiments of Tables 6 to 7, Table 10 below may be used as an input and an output for training a prediction model for the PC ‘Na.’ An output value through an operation of the prediction model for the PC ‘Na’ is a value between 0 and 1, and training may be performed to reduce a difference between the output value and an output value in the right column of Table 10. According to one embodiment, when dimension reduction using PCA is performed in Step S33, a dimensionally reduced feature vector may be used as input data.

TABLE 10

| Type of | Training input | Training output |
|---|---|---|
| substance | data (value) | data (value) |
| Substance 0 | (1 1 0 0) | 1 |
| Substance 1 | (1 0 0 1) | 0 |
| Substance 2 | (0 0 1 0) | 1 |
| Substance 3 | (0 1 0 1) | 1 |

In the embodiments of Tables 6 to 7, Table 11 below may be used as an input and an output for training a prediction model for the PC ‘Da.’ An output value through an operation of the prediction model for the PC ‘Da’ is a value between 0 and 1, and training may be performed to reduce a difference between the output value and an output value in the right column of Table 11. According to one embodiment, when dimension reduction using PCA is performed in Step S33, a dimensionally reduced feature vector may be used as input data.

TABLE 11

| Type of | Training input | Training output |
|---|---|---|
| substance | data (value) | data (value) |
| Substance 0 | (1 1 0 0) | 1 |
| Substance 1 | (1 1 0 1) | 1 |
| Substance 2 | (0 0 0 0) | 0 |
| Substance 3 | (0 1 0 0) | 0 |

In the embodiments of Tables 6 to 7, Table 12 below may be used as an input and an output for training a prediction model for the PC ‘Ra.’ An output value through an operation of the prediction model for the PC ‘Ra’ is a value between 0 and 1, and training may be performed to reduce a difference between the output value and an output value in the right column of Table 12. According to one embodiment, when dimension reduction using PCA is performed in Step S33, a dimensionally reduced feature vector may be used as input data.

TABLE 12

| Type of | Input data | Output data |
|---|---|---|
| substance | (value) | (value) |
| Substance 0 | (0 1 0 0) | 0 |
| Substance 1 | (1 1 0 1) | 1 |
| Substance 2 | (0 0 1 0) | 1 |
| Substance 3 | (0 1 0 0) | 0 |

Referring back to FIG. 1, in Step S1500, the computing device may obtain a feature vector of a new drug candidate substance, input the obtained feature vector to each of the pharmacological effect prediction models by PC created in Step S34 of FIG. 3, and obtain an output value.

FIG. 5 is a detailed flowchart of the method performed in Step S1500 of FIG. 1, according to one embodiment.

In Step S1520, the computing device may calculate similarity between the new drug candidate substance and substances for which the PCs are known. If the number of the substances for which the PCs are known is K, a similarity adjacency matrix having a size of 1×K may be obtained. If there are 2886 types of substances for which the PCs are known, a similarity adjacency matrix corresponding to a specific structural similarity type has a size of 1×2886. In the similarity adjacency matrix, substances for which the PCs are known may be disposed on a horizontal axis and the new drug candidate substance is disposed on a vertical axis, and a similarity value calculated based on a predetermined structural similarity type may be displayed at a point at which the horizontal axis and the vertical axis intersect. A structural similarity type, which is a reference for calculating similarity, is what a user selects in Step S1300 of FIG. 1. For example, the structural similarity type may be one of the eight similarity types in Table 1.

In Step S1530, the computing device may obtain a feature vector of a new drug candidate substance by converting a similarity value to a binary value using a threshold value. The obtained binary vector may be obtained as a feature vector by converting the obtained binary vector to 1 if each of values of the obtained similarity adjacency matrix having a size of 1×K is greater than or equal to a predetermined threshold value, and by converting the same to 0 if it is less than the threshold value. The threshold value for obtaining a binary vector is equal to the threshold value used in Step S32 of FIG. 3.

According to one embodiment, the feature vector of the new drug candidate substance may be subjected to dimension reduction. The dimension reduction method is the same as the method described above in Step S33 of FIG. 3. The principal component analysis (PCA) technique used in Step S33 may be applied for the dimension reduction of the feature vector of the new drug candidate substance. As an example, the computing device may perform vector product on an eigen vector derived by applying the PCA in Step S33 of FIG. 3 by a specific vector of Step S1530. A dimensionally reduced feature vector may be obtained based on a vector product result. Through the above-described process, the feature vector of the new drug candidate substance may be dimensionally reduced to a vector having a size of 1×2, which is composed of Principal Component 1 (PC1) and Principal Component 2 (PC2).

In Step S1540, the computing device may obtain an output value by inputting the feature vector of the new drug candidate substance to the pharmacological effect prediction models by PC prepared in Step S1400 of FIG. 1. If an output value of a pharmacological effect prediction model for a current PCⁱ(0<i≤N, N is the number of PC types) is greater than or equal to the reference value (REF) (Yes), it may be predicted that the new drug candidate substance has the PCⁱ (Step S1550), and if otherwise (No), it may be predicted that the new drug candidate substance does not have the PCiⁱ (Step S1560). The reference value (REF) may be 0, but is not limited thereto.

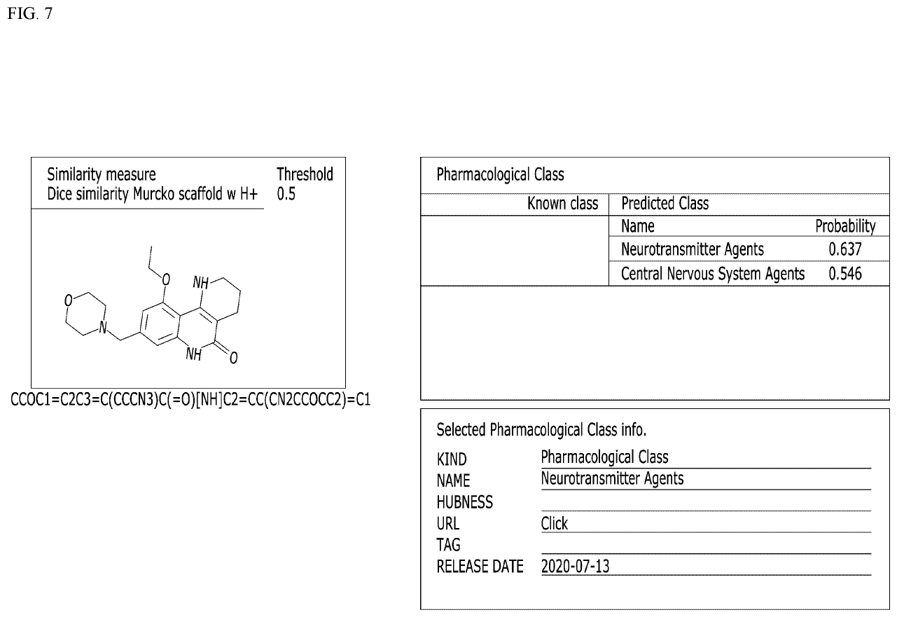

FIG. 7 shows a screen on which a value for the probability that a new drug candidate has a specific PC is output, according to one embodiment. Referring to FIG. 7, the computing device may respectively output output values of 0.637 and 0.546 by inputting the new drug candidate substance CCOC1=C2C3=C(CCCN3)C(═O)[NH]C2=CC(CN2CCOCC2)=C1 into pharmacological effect prediction models of PC=Neurotransmitter Agents and PC=Central Nervous System Agents, respectively.

Meanwhile, the above-described method for predicting pharmacological effects of a new drug candidate substance may be implemented as a computer-readable code on a computer-readable recording medium. The computer-readable recording medium includes all types of recording devices in which data readable by a computer system is stored. Examples of the computer-readable recording medium include ROMs, RAMS, CD-ROMs, magnetic tapes, floppy disks, optical data storage devices, etc., and also include those implemented in the form of transmission over the Internet. In addition, the computer-readable recording medium is distributed in a computer system connected through a network, so that a processor-readable code may be stored and executed in a distributed manner.

The descriptions are intended to provide exemplary configurations and operations for implementing the present invention. The technical spirit of the present invention will include not only the embodiments described above, but also implementations that can be obtained by simply changing or modifying the above embodiments. In addition, the technical spirit of the present invention will also include implementations that can be achieved by easily changing or modifying the embodiments described above in the future.

1. A method for predicting pharmacological effects of a new drug candidate substance performed by a computing device, the method comprising:
receiving information on a new drug candidate substance;
selecting a structural similarity type, which is a reference for determining the similarity between substances;
preparing pharmacological effect prediction models corresponding to the selected structural similarity type from among a plurality of pharmacological effect prediction models created by structural similarity type and pharmacological class; and
predicting whether the new drug candidate substance will have a pharmacological class corresponding to each of the pharmacological effect prediction models based on an output value obtained by inputting information on the new drug candidate substance into each of the prepared pharmacological effect prediction models,
wherein:
the structural similarity type is further classified according to which calculation method between a Dice similarity calculation method and a Tanimoto similarity calculation method is to be applied, whether a Bemis-Murcko scaffold is applied, and whether a hydrogen atom bond is applied;
each of the plurality of pharmacological effect prediction models created by structural similarity type and pharmacological class is created based on machine learning using substances already known whether to have a specific pharmacological class;
in each of the pharmacologic effect prediction models, a binary vector obtained by using a threshold value for binarization is input as a feature vector of the new drug candidate substance to a similarity calculated according to the selected structural similarity type between the new drug candidate substance and the already known substance;
the predicting step predicts:
if a first output value obtained by inputting the feature vector into a first pharmacological effect prediction model corresponding to a first pharmacological class is greater than or equal to a reference value, that the new drug candidate substance has the first pharmacological class;
if the first output value is less than the reference value, that the new drug candidate substance does not have the first pharmacological class;
if a second output value obtained by inputting the feature vector into a second pharmacological effect prediction model corresponding to a second pharmacological class is greater than or equal to the reference value, that the new drug candidate substance has the second pharmacological class; and
if the second output value is less than the reference value, that the new drug candidate substance does not have the second pharmacological class.

2. The method of claim 1, comprising further creating the plurality of pharmacological effect prediction models by structural similarity type and pharmacological class, wherein the creating of the plurality of pharmacological effect prediction models includes creating a pharmacological effect prediction model corresponding to a specific pharmacological class, wherein the creating of the pharmacological effect prediction model corresponding to the specific pharmacological class includes:
creating a two-dimensional adjacency matrix in which substances known whether to have the specific pharmacological class are disposed on each of a horizontal axis and a vertical axis, and similarity between two substances calculated on the basis of one of the structural similarity types is displayed at a point at which the horizontal axis and the vertical axis intersect;
obtaining a binary adjacency matrix by converting the similarity to 1 if the similarity is greater than or equal to the threshold value and at least one of the two substances is known to have the specific pharmacological class, and by converting the similarity to 0 if the similarity is less than the threshold value or if both the two substances are known not to have the specific pharmacological class;
obtaining values listed in each of rows constituting the binary adjacency matrix as a feature vector of the known substances corresponding to each of the rows; and
creating a pharmacological effect prediction model corresponding to one of the structural similarity types and the specific pharmacological class by applying the feature vector of the known substances and whether each of the known substances has the specific pharmacological class as an input and an output, respectively to perform training.

3. The method of claim 2, wherein the creating of the pharmacological effect prediction model corresponding to the specific pharmacological class further comprises reducing the dimension of the binary adjacency matrix by applying a principle component analysis (PCA) technique, wherein the feature vector of the known substances is obtained from the dimensionally reduced binary adjacency matrix.

4. The method of claim 2, further comprising obtaining the feature vector of the new drug candidate substance that is input into the pharmacological effect prediction model, wherein the obtaining of the feature vector of the new drug candidate substance includes:
calculating similarity between the new drug candidate substance and the known substances on the basis of the selected structural similarity type; and
obtaining, as the feature vector of the new drug candidate substance, a binary vector obtained by converting the similarity to 1 if the similarity is greater than or equal to the threshold value, and by converting the similarity to 0 if the similarity is less than the threshold value.

5. The method of claim 3, further comprising obtaining the feature vector of the new drug candidate substance that is input into the pharmacological effect prediction model, wherein the obtaining of the feature vector of the new drug candidate substance includes:
calculating similarity between the new drug candidate substance and the known substances on the basis of the selected structural similarity type;
obtaining a binary vector by converting the similarity to 1 if the similarity is greater than or equal to the threshold value, and by converting the similarity to 0 if the similarity is less than the threshold value; and
obtaining, as the feature vector of the new drug candidate substance, a result obtained by performing vector multiplication on an eigen vector derived by applying the principle component analysis (PCA) technique by the binary vector.
